(19) United States
(12) Patent Application Publication
(10) Pub. No.: US 2014/0030669 A1
(43) Pub. Date: Jan. 30, 2014

(54) Surgical Instrument Having Integrated Navigation Control

(71) Applicants: Joachim Hey, Konigswinter (DE); Manfred Breuer, Alfter (DE); Nils Hanssen, Bonn (DE)

(72) Inventors: Joachim Hey, Konigswinter (DE); Manfred Breuer, Alfter (DE); Nils Hanssen, Bonn (DE)

(21) Appl. No.: 14/001,233

(22) PCT Filed: Dec. 28, 2011
(86) PCT No.: PCT/EP11/74192

§ 371 Date: Oct. 11, 2013

(30) Foreign Application Priority Data
Feb. 25, 2011 (DE) 10 2011 012 460.8

(51) Int. Cl. A61C 1/08; A61B 1/00; A61B 1/04; A61C 19/04; A61B 1/24

(52) U.S. Cl. CPC A61C 1/082; A61C 19/04; A61B 1/24; A61B 1/04; A61B 1/00165; A61B 1/00188; A61B 1/00193; A61B 1/00045
USPC 433/27

(57) ABSTRACT

The invention relates to a system for carrying out a treatment of a human or animal body by a surgeon, comprising a hand-held instrument (2) having an instrument head (3) that acts on an operating field of the body and supports a treatment tool (12). The system also comprises a computer (4) on which a navigation program is provided to assist the guidance of the instrument head (3), wherein body data representing the part of the body containing the operating field, planning data representing the planned treatment, and instrument data representing the position and orientation of the instrument head (3) are available to the navigation program. A positioning means for recording the instrument data is present, wherein the navigation program compares the instrument data as actual data with the planning data as desired data, and a signalling means (10) is provided, which indicates to the surgeon a deviation of the actual data from the desired data. The positioning means comprises an image-recording means that is located at the instrument head (3) and that records, during the handling of the instrument head and in particular in rapid sequence, single images of a body part that is represented in the body data and is in a defined relationship with the treatment site, wherein the orientation of the image-recording means is in a defined relationship with the instrument head, and the navigation program generates the instrument data by matching the single images with the body data.

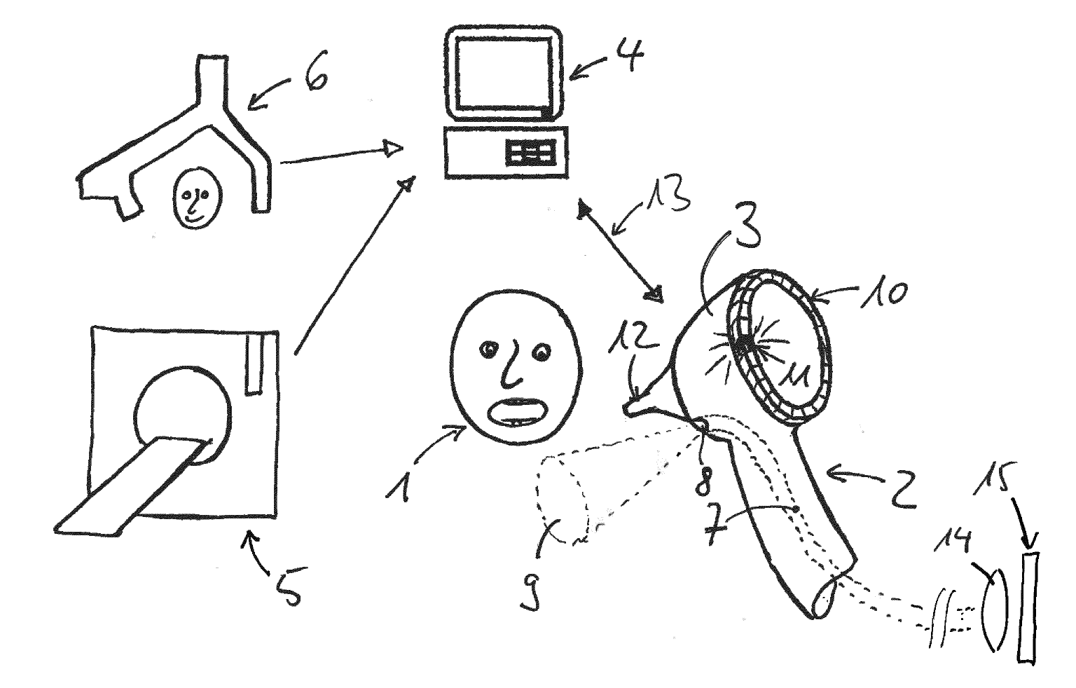

The present invention relates to a system for carrying out a treatment of a body, in particular a human or animal body, comprising a hand-held instrument having an instrument head that acts on the body and supports a tool, and comprising a control unit having a computer on which a navigation program is provided to assist the guidance of the instrument head, wherein body data representing the part of the body containing the operating field, planning data representing the planned treatment, and instrument data representing the position and orientation of the instrument head are available to the navigation program, wherein a positioning means for recording the instrument data is present, wherein the navigation program compares the instrument data (“actual data”) to the planning data (“desired data”), wherein a signalling means is provided, which indicates to the surgeon a deviation of the actual data from the desired data.

Such a system for surgical interventions on the jaw in particular, with which a movement of a dental drill in the treatment region can be detected, is known from DE 102 59 250 A1. This uses an externally positioned positioning means in the manner of a camera system in order to determine the current position of the instrument head. A similar system, which likewise uses such complex positioning means, is presented in DE 101 10 093 A1. A particular problem of the known systems is the registration, that is to say the transformation, of the actual data into the system of the desired data. In order to perform this registration process, the systems use specific marker structures, which on the one hand are attached the instrument and on the other hand are attached to the patient. The position and orientation of these marker structures in space is determined via the externally positioned positioning means and is projected into the planning data. The position of the patient and the position of the instrument can thus be determined by means of the marker structures and can be incorporated as actual data into the planning data (desired data). Deviations between current data and desired data are indicated to the treating doctor in accordance with DE 102 50 250 A1 directly at the instrument head or, in accordance with DE 101 10 093 A1, on an external screen.

These systems, due to the complex structure, are problematic in terms of the mounting of the markers, since the marker structures have to protrude from the treatment region in order to be detected by the external optical positioning means. Due to the consideration of the marker structures, the working range is also severely restricted. In addition, complex calibration and set-up of the system is necessary, and not only before initiation of an intervention. Conditions that change during the intervention also sometimes require repeated calibration.

Besides these technically complex systems, individually fabricated templates are also known from the dental field, which have a drilling channel, of which the drilling depth is limited, for the dental drill to be guided by hand. The disadvantage of these drilling templates however is that they each have to be fabricated individually in complex process steps.

The object of the present invention is now to create a system, which can be used particularly easily and above all without the aid of external positioning means, and which ensures exact control of the instrument, and with which the planning is to be implemented. The system is also to be usable in a versatile manner for the implementation of planning procedures.

This object is achieved by a system having the characterising features of claim 1.

The underlying concept of the invention lies in attaching the positioning means directly on the instrument head, such that the instrument does not have to be positioned by external means, but that the instrument itself makes “an image” of its environment, wherein this image is then compared with the desired data.

To record the single images, this positioning means attached directly to the instrument head has a means for recording images, which enables the single images to be recorded during the handling of the instrument head. From at least two single images, which are recorded simultaneously from different perspectives or in rapid succession after a movement, it is possible to calculate coordinates in three dimensions and therefore stereoscopic images. Here, to obtain a high sensitivity of the system over time, it is advantageous to record a large number of such single images in rapid succession in the manner of a film sequence. In a particularly simple and therefore advantageous embodiment, such an image-recording means generates images in the optical field. It is also conceivable however to record images by means of other media, in particular by means of ultrasound.

The single images do not necessarily have to originate directly from the treatment site. For the guidance according to the invention of the instrument head in the coordinate system of the planning data, it is thus merely necessary for the single images to show a body part that is represented in the body data and that is in a defined relationship with the treatment site. For example, the image-recording means can be oriented on the tooth, adjacent to the tooth gap to be treated, that has its equivalent in the body data and therefore in the planning data. It is also key here that the orientation of the image-recording means is in a defined, unalterable relationship with the instrument head, such that the single images are accordingly in a defined relationship with the treatment site.

It is possible for the recording means to be directed directly to the treatment site and for single images of the treatment site and the immediate environment to be recorded. By comparing the single images with the body data, the navigation program can generate the current instrument data. As mentioned above, the single images are advantageously recorded in rapid succession, such that the movement of the instrument head can be projected as instrument data into the body and planning data. After comparing the actual data with the desired data originating from the planning, the deviation from the planning is indicated to the surgeon on the signalling means, such that he can correct his movement accordingly.

In accordance with the invention, the image reconstructed from the recorded actual data and presented by the recording means is compared in relation to the image representing the desired data which should be presented by the recording means. As a result of the efforts of the navigation program, which attempts to line up the two images, the handling instruction illustrated on the signalling means is indicated to the surgeon. If he follows this instruction, the instrument ultimately reaches the correct position. The information concerning the direction in which the surgeon has to move his instrument based on the planning is available in particular directly at the location of the event, such that a particularly ergonomic mode of operation is possible.

Here, the recorded image data is conveyed to a computer which is associated with the system and which identifies the structures on the basis of the surfaces and calculates the location and position of the instrument with respect to this surface. At the same time, the position and location of the planned intervention with respect to this surface is known. Now, the computer can give control commands to correct the position and location of the instrument until this corresponds to the planned intervention.

Here, it is particularly advantageous if the signalling means is attached to the instrument in such a way that the surgeon can observe the signalling means whilst he can see the instrument head. The surgeon is thus provided during the handling of the instrument with information concerning the deviations from the planning without losing sight of the manually guided instrument. This information provides the surgeon with the opportunity to correct the instrument guidance under the direct control of the system.

In accordance with this embodiment, the position and the location of the instrument with respect to the planning is visualised on the instrument itself, such that this information is not indicated to the surgeon on external screens and displays located outside the treatment region. Such an optical display on the instrument itself therefore enables an intuitive operation by the surgeon without the surgeon having to turn his view away from the site of the intervention. The visualisation guides the surgeon's hand so to speak in the correct direction. With a system equipped in accordance with the invention, the manual navigation of the instrument, which in particular is a dental drill, can be performed exactly in accordance with the treatment planning carried out previously. The instrument can be guided manually in direct orientation relative to the object in the vicinity of which the intervention takes place. This is possible since the treatment planning occurs with respect to the rigid structure of the object in the environment of the treatment site.

The field of vision is advantageously directed to the operating field and in particular also to the instrument head, and it is thus possible for the current image of the image-recording means to be displayed (additionally) on a screen visible to the surgeon. In a particularly advantageous embodiment, the positioning means comprises at least two image-recording means in order to enable exact orientation in all three dimensions. For example, the two positioning means can be arranged such that they are arranged relative to one another in a manner suitable for generating a stereoscopic recording of the body part. They may also be directed to different regions however, since the orientation in space can also be established from the comparison of two different images. It is merely key that the arrangement is defined and the system is calibrated to this defined arrangement.

In order to ensure an exact orientation of the instrument, it is advantageous if the navigation program, for comparison of the single images or of the film with the body data, filters out from the body data solid structures, such as exposed bone and/or teeth. These structures are particularly suitable as fixed and permanent reference points.

Since the scans of the surface of a structure constitute particularly representative and easily processed recorded images, it is advantageous if the image-recording means has a corresponding device, for example a laser scanner, with which such a surface scan can be carried out over the body part. Such a device is to be designed such that it creates the scans at high speed, such that a rapid sequence of many single images is possible.

In a simpler embodiment, the system operates as a camera, wherein different designs are possible. On the one hand, the image-recording means may be a light-conducting fibre ending in the instrument head, the camera being located at the other end of said fibre. On the other hand, the camera may be arranged in the instrument itself, in order to detect the surface in the environment of the instrument. It is generally advantageous however if light is guided to the treatment field by means of a light-conducting fibre in the instrument head. On the one hand, the quality of the recorded images is thus considerably improved. On the other hand, the additional light makes it easier for the surgeon to orientate the instrument. In a particularly advantageous embodiment, the image-recording means has a lens optic focussing automatically on the body part.

The treatment planning has been performed on a computer prior to the treatment process, the planning being stored as planning data in the system of the body data. Here, the body data can be created on the basis of sectional images, which are generated by a computer tomograph (CT), a magnetic resonance scanner (MR), or a dedicated dental 3D imaging device. In the case of dental treatment, the exact location of the drilling channel and the correct depth thereof can be predetermined within the scope of the planning. The instrument data are raised here as actual data by the positioning means. The coordinate system of the body data (body coordinate system, KOS) is brought into conformity with the coordinate system of the actual treatment (OP-KOS) within the scope of the registration process. The location of the instrument is detected by the positioning means and is projected virtually into the body data also containing the planning data. The instrument is visualised in the system of the body data in relation to the body.

An exemplary embodiment of the invention is illustrated in the FIGURE and is described in greater detail hereinafter.

The FIGURE shows a system for carrying out treatment on a jaw or tooth, said treatment process being carried out on a patient 1. The system firstly comprises a dental drill 2 as an instrument that can be held by the doctor, having an instrument head 3 acting on the jaw and supporting a drill. In addition, the system comprises a control unit having a computer 4, on which a navigation program for guiding the instrument head 3 is provided. The dental drill 2 is connected via a data line 13 to the computer 4. The navigation program accesses body data, which are generated by a CT 5 or a dental 3D imaging device 6 and on the basis of which the planning has been performed.

Prior to the intervention, planning data are recorded by means of one of the devices 5 or 6, said data forming a basis for the navigation program. The images required for the treatment planning can be generated both prior to the intervention and during the intervention. They can be recorded by means of imaging methods using different technologies, such as X-ray, magnetic resonance, ultrasound and/or optically. The planning is likewise carried out on the computer 4. During the treatment process, the location of the instrument head within the body data is determined and is compared with the planning data. For the display of deviations, a signalling means 10 is provided on the dental drill 2 and indicates to the surgeon any deviations of the instrument data from the planning data, wherein the signals are perceptible whilst the surgeon has the instrument head in his direct field of vision.

The positioning means has an optical waveguide 7, which runs into an eye 8 in the vicinity of the instrument head 12. With this eye 8, the positioning means has a field of vision 9, in which the treatment region is located. The optical waveguide 7 leads out from the dental drill 2 to a recording means, which comprises a lens 14 and an electronic camera 15. There, each single image is detected digitally and is transmitted to the navigation program. The instrument data are thus detected in a coordinate system spanning the space of the body data. Both the imaging devices 5 and 6 and also the recording means are each connected via a data line to the computer 4 and transmit the body data and instrument data respectively via these lines.

As is shown in the FIGURE, the drill has LEDs 10 as signalling means on the drill head, said LEDs being arranged in the form of a ring. A pointer which has the function of a compass needle pointing in the correct direction can be provided via a varying luminosity of the lamps arranged in the ring. The LEDs 10 are controlled via the computer 4, which also receives the previously stored planning data and which, as described, is connected to the navigation system. The doctor first positions the tip 12 of the drill 2, wherein he is instructed of the direction by the red flashing LED 11 of the ring 10. The information necessary for navigation is ascertained by the computer, as described above, from the planning data and the location of the instrument received by the computer from the navigation system. If the surgeon has reached the position within the scope of a defined tolerance, the entire outer LED ring lights up. He then sets the correct orientation of the drill by tilting the drill 2 in the direction that is now visualised to him by a blue, lit LED on the ring 10. If the position of the tip and the location in space are now correct, the colour of the entire outer ring changes and is lit in green. The doctor can now drill, since the drill is oriented exactly in accordance with the planning. Once he has reached the correct depth, the ring flashes. An acoustic signal can additionally be given by the computer.

1. A system for carrying out a treatment of a human or animal body by a surgeon, comprising a hand-held instrument (2) having an instrument head (3) that acts on an operating field of the body and supports a treatment tool (12), and comprising a computer (4) on which a navigation program is provided to assist the guidance of the instrument head (3), wherein body data representing the part of the body containing the operating field, planning data representing the planned treatment, and instrument data representing the position and orientation of the instrument head (3) are available to the navigation program, wherein a positioning means for recording the instrument data is present, wherein the navigation program compares the instrument data as actual data with the planning data as desired data, wherein a signalling means (10) is provided, which indicates to the surgeon a deviation of the actual data from the desired data,
characterised in that
the positioning means comprises an image-recording means that is located at the instrument head (3) and that records, during the handling of the instrument head and in particular in rapid sequence, single images of a body part that is represented in the body data and is in a defined relationship with the treatment site, wherein the orientation of the image-recording means is in a defined relationship with the instrument head, wherein the navigation program generates the instrument data by matching the single images with the body data.

2. The system according to claim 1, characterised in that the signalling means (10) is provided on the instrument head (3), wherein the signalling means (10) indicates to the surgeon the deviation of the actual data from the desired data whilst the instrument head (3) is in his field of vision, wherein the instrument is a dental drill (2) in particular.

3. The system according to claim 1, characterised in that the positioning means has two image-recording means, which are oriented relative to one another in a manner suitable for generating a stereoscopic recording of the body part.

4. The system according to claim 1, characterised in that the navigation program, for comparison of the single images, filters out from the body data solid structures, such as exposed bone and/or teeth.

5. The system according to claim 1, characterised in that the field of vision (9) of the image-recording means is directed to the operating field and in particular also to the instrument head or the treatment tool (12).

6. The system according to claim 5, characterised in that the current image of the image-recording means is displayed on a screen visible to the surgeon.

7. The system according to claim 1, characterised in that the image-recording means has a device that carries out surface scans over the body part and generates the single images therefrom.

8. The system according to claim 1, characterised in that the image-recording means is a light-conducting fibre (7) ending in the instrument head, at the other end of which a camera (15) is located.

9. The system according to claim 1, characterised in that light can be guided to the operating field by means of a light-conducting fibre (7) ending in the instrument head.

10. The system according to claim 1, characterised in that the image-recording means has a lens optic (14) focussing automatically on the body part.
